(19) United States
(12) Patent Application Publication
(10) Pub. No.: US 2019/0321484 A1
(43) Pub. Date: Oct. 24, 2019

(54) ANTIBODY DRUG CONJUGATES

(71) Applicant: Hoffmann - La Roche Inc., Little Falls, NJ (US)

(72) Inventors: Gaston Hubertus Maria VONDENHOFF, Munich (DE); Stefan TRENKER, Munich (DE)

(73) Assignee: Hoffmann - La Roche Inc., Little Falls, NJ (US)

(21) Appl. No.: 16/462,274

(22) PCT Filed: Nov. 21, 2017
(86) PCT No.: PCT/EP2017/079869

§ 371 Date: May 20, 2019

(30) Foreign Application Priority Data
Nov. 22, 2016 (EP) 16200071.5

(51) Int. Cl. A61K 47/68

(52) U.S. Cl. CPC A61K 47/6889; A61K 47/6855

(57) ABSTRACT

The present invention pertains to an antibody-drug conjugate comprising an antibody, ferric iron, and at least one drug molecule, and to a pharmaceutical composition comprising the antibody-drug conjugate. The invention further relates to the use of the antibody-drug conjugates in the treatment of diseases, e.g. cancer.

This application corresponds to the U.S. national phase of International Application No. PCT/EP2017/079869, filed Nov. 21, 2017, which, in turn, claims priority to European Patent Application No. 16.200071.5 filed Nov. 22, 2016, the contents of which are incorporated by reference herein in their entirety.

### FIELD OF THE INVENTION

The present invention relates to the use of antibody-drug conjugates in the treatment of diseases such as cancer.

### BACKGROUND

Traditional cancer chemotherapy is often accompanied by systemic toxicity to the patient. Targeted therapy approaches seek to specifically interfere with molecular targets and pathways that are important for the proliferation of cancer cells. These targets are preferentially expressed either intracellularly or on the surface of tumor cells. Thus, targeted therapy offers the potential to generate agents that will be selectively cytotoxic to tumor cells, coupled with lower toxicity to the host, resulting in a larger therapeutic index. One approach was the development of inhibitors of tyrosine kinases that are misregulated and/or overexpressed in cancer cells. Another targeted therapy approach is the use of small molecules that selectively bind to the surface of tumor cells to deliver a cytotoxic compound (e.g. a folate-vinca alkaloid conjugates).

Monoclonal antibodies against antigens on cancer cells offer an alternative tumor-selective treatment approach. However, many monoclonal antibodies are not sufficiently potent to be therapeutically active on their own. Antibodydrug conjugates (ADCs) use antibodies to deliver a potent cytotoxic compound selectively to tumor cells, thus improving the therapeutic index of chemotherapeutic agents. Two of the most prominent examples are the approved drugs ado-trastuzumab emtansine (Kadcyla®) and brentuximab vedotin (Adcetris®).

Current ADCs, however, often suffer from insufficient potency of the ADC as compared to the parent free drug. This may be due to insufficient cleavage from the antibody after the ADC is being taken up by the cancer cell and processed in the endosome/lysosome. Current ADCs often need to be linked to the antibody using a specialized linker that cannot or only partially be cleaved off during intracellular metabolism.

Another drawback of certain ADCs is their poor stability in the blood. Labile linkers release the drug too early, which results in loss of activity of the ADC and increased negative side-effects. On the other hand, highly stable linkers hamper efficient release once the ADC reaches the endosome.

In addition, it is generally difficult to achieve a sufficiently high intracellular concentration of cytotoxic compound, as the number of antigens on a cancer cell is typically ≤10⁵. However, the intracellular concentration of cytotoxic compound should preferably be significantly higher than 10⁵.

WO 2013/103707 A1 discloses various ADCs comprising a platinum cation.

Thus, there is a need for improved antibody drug conjugates overcoming one or more of the disadvantages of existing therapies.

### SUMMARY OF THE INVENTION

The inventors of this application found that ADCs in which the drug is linked to the antibody via ferric iron are stable and selective, and can transport a high number of drug molecules into the target cells. In addition, the intracellular release of the active agent is improved due to reduction of the iron cation only within the endosomes. Undesired release of the drug in the blood stream is thereby prevented or at least substantially reduced.

The present invention therefore relates to, but is not limited to, the embodiments defined in the following items [1] to [43]:
[1] An antibody-drug conjugate comprising an antibody, a ferric iron bound to the antibody, and at least one drug molecule bound to the ferric iron.
[2] The antibody-drug conjugate of item [1], wherein the ferric iron is complexed by the antibody and the drug molecule.
[3] The antibody-drug conjugate of item [1] or [2], wherein the ferric iron is bound to the antibody via a first linker.
[4] The antibody-drug conjugate of item [3], wherein the first linker comprises an iron complexing group capable of binding to the ferric iron.
[5] The antibody-drug conjugate of item [3] or [4], wherein the first linker is covalently bound to the antibody.
[6] The antibody-drug conjugate of any one of the preceding items, wherein at least two drug molecules are bound to the ferric iron.
[7] The antibody-drug conjugate of any one of the preceding items, wherein the average number of drug molecules per antibody in said conjugate is at least 5.
[8] The antibody-drug conjugate of any one of the preceding items, wherein the average number of drug molecules per antibody in said conjugate is at least 10.
[9] The antibody-drug conjugate of any one of the preceding items, wherein the average number of drug molecules per antibody in said conjugate is at least 15.
[10] The antibody-drug conjugate of any one of the preceding items, wherein the drug molecule is not released from the antibody-drug conjugate when the antibody-drug conjugate is in human blood.
[11] The antibody-drug conjugate of any one of the preceding items, wherein the drug molecule is released from the antibody-drug conjugate when the antibody-drug conjugate is in the endosomal compartment of a human cell.
[12] The antibody-drug conjugate of any one of the preceding items, wherein the drug molecule is selected from the group consisting of anti-cancer agents, anti-inflammatory agents, and anti-infective agents.
[13] The antibody-drug conjugate of any one of the preceding items, wherein the drug molecule is an anti-cancer agent.
[14] The antibody-drug conjugate of item [13], wherein the anti-cancer agent is selected from the group consisting of microtubule structure formation inhibitors, meiosis inhibitors, RNA polymerase inhibitors, topoisomerase inhibitors, DNA intercalating agents, DNA alkylating agents, and ribosome inhibitors.
[15] The antibody-drug conjugate of any one of the preceding items, wherein the antibody is capable of binding to an antigen expressed on a tumor cell.
[16] The antibody-drug conjugate of any one of the preceding items, wherein the drug molecule comprises an iron complexing group which binds to the ferric iron.
[17] The antibody-drug conjugate of any one of items [1] to [15], wherein the drug molecule is covalently linked to an iron complexing group which binds to the ferric iron.
[18] The antibody-drug conjugate of item [16] or [17], wherein said iron complexing group is selected from the group consisting of pyridine derivatives, a hydroxamate group, a catecholate group, a carboxylate group, and acids thereof.
[19] The antibody-drug conjugate of any one of the preceding items, having the structure

Ab[-L1-Me (-L2-D)ₙ]ₘ,
wherein
   Ab is the antibody,
   L1 is a first linker,
   Me is the ferric iron,
   L2 is a second linker or absent,
   D is the drug molecule,
   m is a number ranging from 1 to 10, and
   n is a number ranging from 1 to 3.
[20] The antibody-drug conjugate of item [19], wherein m is a number ranging from 2 to 6.
[21] The antibody-drug conjugate of item [19], wherein m is a number ranging from 3 to 5.
[22] The antibody-drug conjugate of any one of items [19] to [21], wherein n is 2 or 3.
[23] The antibody-drug conjugate of any one of the preceding items, wherein the drug molecule is released from the ferric iron if the ferric iron is reduced.
[24] The antibody-drug conjugate of any one of the preceding items, wherein the drug molecule is released from the ferric iron at a pH of less than 5.
[25] The antibody-drug conjugate of any one of the preceding items, which is stable at pH 8.
[26] The antibody-drug conjugate of any one of the preceding items, which is stable at pH 7.
[27] The antibody-drug conjugate of any one of the preceding items, which is stable at pH 6.
[28] The antibody-drug conjugate of any one of the preceding items, which is stable at pH 5.
[29] A pharmaceutical composition comprising the antibody-drug conjugate of any one of the preceding items.
[30] The pharmaceutical composition of item [29], further comprising a pharmaceutically acceptable excipient.
[31] The antibody-drug conjugate of any one of items [1] to [28] for use as a therapeutic agent.
[32] The antibody-drug conjugate of any one of items [1] to [28] for use in the treatment of a disorder.
[33] The antibody-drug conjugate of any one of items [1] to [28] for use in the treatment of cancer.
[34] The antibody-drug conjugate for use according to item [33], wherein said treatment comprises administering to a patient the antibody-drug conjugate of any one of items [1] to [28] and an anti-cancer agent different from said antibody-drug conjugate.
[35] Use of ferric iron for preventing or reducing the release of a drug molecule from an antibody-drug conjugate in blood, wherein the ferric iron is complexed by the antibody and the drug molecule to form the antibody-drug conjugate.
[36] The use of item [35], wherein the drug molecule is released from the antibody-drug conjugate in the endosomal compartment of a cell.
[37] The use of item [35] or [36], wherein the antibody-drug conjugate is the antibody-drug conjugate as defined in any one of items [1] to [28].
[38] Use of the antibody-drug conjugate of any one of items [1] to [28] for preventing or reducing the release of a drug molecule from an antibody-drug conjugate in blood.
[39] A method of preventing or reducing the release of a drug molecule from an antibody-drug conjugate in blood, comprising complexing ferric iron with the antibody and the drug molecule to form the antibody-drug conjugate.
[40] The method of item [39], wherein the antibody-drug conjugate is the antibody-drug conjugate as defined in any one of items [1] to [28].
[41] Use of ferric iron for linking a drug molecule to an antibody.
[42] The use of item [41], wherein an antibody-drug conjugate is obtained by said linking.
[43] The use of item [41] or [42], wherein said antibody-drug conjugate is the antibody-drug conjugate as defined in any one of items [1] to [28].

### BRIEF DESCRIPTION OF THE DRAWINGS

FIG. 1. A New Concept for ADCs: Fe-Complex as a pH/Fe-reductase labile linker. The cytotoxic compound is conjugated to ligands that complex iron. The complex is then conjugated to an antibody that has its natural receptor on the cancer cell. Upon antibody-antigen binding the entire ADC is taken up by endocytosis and the complex disintegrates inside the endosome due to low pH and/or Fe-reductase activity, releasing the drug. Further action of, for example peptidases, may be required to release the active entity.

FIGS. 2A and 2B. The iron may be complexed by the cytotoxic drug itself. In this example, histone deacetylase inhibitors (e.g. SAHA (Vorinostat)) are used to complex iron. This is an example for traceless drug release of iron-based ADCs. The hydroxamate functionality enables iron complexation through the drug itself without any modification of the molecule.

FIGS. 3A and 3B. The very potent drug Doxorubicin may also be used to complex iron after some modification of the amine function. Further mode of action is as described in FIG. 1.

FIGS. 4A and 4B. The upper graph (FIG. 4A) shows analytical LC-runs of complex A at pH 5.0, at 37° C., over 16 h. The lower graph (FIG. 4B) shows analytical LC-runs of complex A at pH 8.0, at 37° C., over 16 h (see example 1).

FIG. 5. Overlay of analytical HPLC chromatograms of ligands used in complexes A and B, their respective iron-complexes, and an additional sample of preformed complex A with free ligand B, showing no intermediate complexes (example 2).

FIG. 6. Overlay of analytical HPLC chromatograms of free ligand (ST116), its respective complex and the hydrogen-reduced complex yielding the respective free ligand (example 3).

FIG. 7. Conjugation of an iron-complexing moiety to the AB, yielding product AB-1, followed by iron complexation together with two auxiliary ligands (AB-2) (example 4).

FIGS. 8A and 8B. Upper graph (FIG. 8A): Fluorescence measurement of conjugated/complexed AB-2 and the non-conjugated AB. Lower graph (FIG. 8B): Difference in fluorescence of the complexed and non-complexed conjugates (example 4).

FIGS. 9A-9D. Fluorescence spectra of AB-2 (FIG. 9A) and its reduced derivative AB-1 (FIG. 9C), whereby the reduced complex gives ˜20% less fluorescence (FIG. 9D—example 4). FIG. 9B depicts the non-conjugated antibody (AB) in isolation.

FIGS. 10A-10C. Fluorescence spectra of a non-reduced ST119 conjugate (FIG. 10A) and its reduced derivative (FIG. 10B) are presented in FIG. 10C. Fluorescence of N-Hydroxamate Ornithine coupled to Fluorescein amine (ST102) is quenched by Fe³⁺ but regains fluorescence upon reduction of Fe³⁺ to Fe²⁺ (example 5).

### DETAILED DESCRIPTION

In a first aspect, the invention relates to an antibody-drug conjugate comprising an antibody, at least one ferric iron bound to the antibody, and at least one drug molecule bound to the ferric iron.

Antibody

As used herein, the term “antibody” refers to an immunoglobulin (Ig), which is defined as a protein belonging to the class IgG, IgM, IgE, IgA, or IgD (or any subclass thereof), or a functional fragment thereof. In the context of the present invention, a “functional fragment” of an immunoglobulin is defined as antigen-binding fragment or other derivative of a parental immunoglobulin that essentially maintains the antigen binding activity of such parental immunoglobulin. Functional fragments are antibodies in the sense of the present invention even if their affinity to the antigen is lower than that of the parental immunoglobulin. “Functional fragments” in accordance with the invention include F(ab′)₂ fragments, Fab fragments, scFv, dsFv, diabodies, triabodies, tetrabodies and Fc fusion proteins. The F(ab′)₂ or Fab may be engineered to minimize or completely remove the intermolecular disulphide interactions that occur between the CH1 and CL domains. The antibodies of the present invention may be part of bi- or multifunctional constructs. The antibodies of the present invention include, but are not limited to, monoclonal antibodies, human antibodies, humanized antibodies, chimeric antibodies, and anti-idiotypic antibodies.

Preferably, the antibody of the present invention is a monoclonal antibody.

The antibody of the ADC of the present invention preferably specifically binds to an antigen expressed on the surface of a cancer cell.

Drug Molecule

The term “drug molecule” or “payload” as used herein refers to a therapeutic or diagnostic agent. Preferred drug molecules include anti-cancer agents, anti-inflammatory agents, and anti-infective (e.g., anti-fungal, antibacterial, anti-parasitic, anti-viral) agents.

Preferably, the drug molecule of the present invention is an anti-cancer agent. Suitable anti-cancer agents include, but are not limited to, alkylating agents, antimetabolites, spindle poison plant alkaloids, cytotoxic/antitumor antibiotics, topoisomerase inhibitors, photosensitizers, and kinase inhibitors.

Also included in the definition of “anti-cancer agent” are: (i) anti-hormonal agents that act to regulate or inhibit hormone action on tumors such as anti-estrogens and selective estrogen receptor modulators; (ii) aromatase inhibitors that inhibit the enzyme aromatase, which regulates estrogen production in the adrenal glands; (iii) anti-androgens; (iv) protein kinase inhibitors; (v) lipid kinase inhibitors; (vi) antisense oligonucleotides, particularly those which inhibit expression of genes in signaling pathways implicated in aberrant cell proliferation; (vii) ribozymes such as VEGF expression inhibitors and HER2 expression inhibitors; (viii) vaccines such as gene therapy vaccines; topoisomerase 1 inhibitors; (ix) anti-angiogenic agents; and pharmaceutically acceptable salts, acids, solvates and derivatives of any of the above.

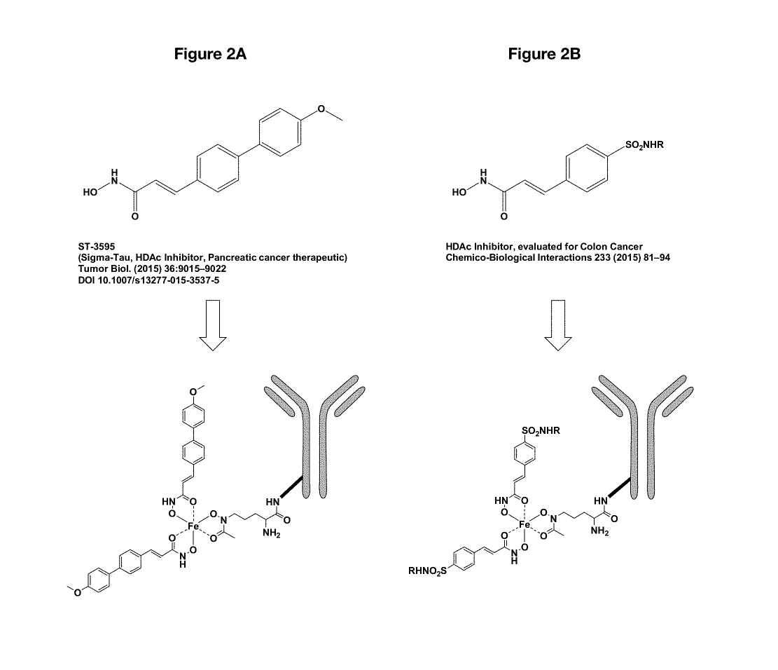

The most preferred anti-cancer agents in the present invention are those that can be removed nearly traceless upon disintegration of the iron-ligand complex. Meaning that compounds with high binding affinities to their target require no or minor modification in order to complex with iron. Most notable in the presented example are compounds such as the HDAc inhibitor ST-3595 (see FIGS. 2A and 2B). However, also different compounds may also be highly suitable to complex with ferric iron while being able to disintegrate upon iron reduction/ligand protonation.

Ferric Iron

The term “ferric iron” as used herein refers to an iron cation with an oxidation number of +3, also designated iron(III), Fe(III) or Fe³⁺.

Generally, pharmaceutically acceptable transition metal cations that are capable of forming a complex with other compounds include, for example, cations of platinum, ruthenium, iridium, scandium, titanium, vanadium, chromium, manganese, iron, cobalt, nickel, copper and zinc. The transition metal cation in the ADC of the invention, however, is ferric iron.

Conjugate

Typically, the ferric iron is indirectly bound to the antibody via a first linker.

The term “linker”, as used herein, refers to a chemical moiety which attaches a molecule or an atom to a chemical compound, e.g. to an antibody or to a drug molecule. Typically, the linker comprises a chain of atoms linked by chemical bonds.

The first linker attaches the ferric iron to the antibody.

One end of the first linker is preferably covalently attached to the antibody. The antibody-reactive end of the first linker is typically a site that is capable of conjugation to the antibody through a cysteine thiol or lysine amine group on the antibody, and so is typically a thiol-reactive group such as a double bond (as in maleimide) or a leaving group such as a chloro, bromo, or iodo, or an R-sulfanyl group, or an amine-reactive group such as a carboxyl group. When the term “linker” is used in describing the linker in conjugated form, one reactive end will be absent (such as the leaving group of a thiol-reactive group) or incomplete (such as there being only the carbonyl of the carboxylic acid) because of the formation of the bonds between the linker and the antibody. Various linker types are described in McCombs et al. (2015) The AAPS Journal, Vol. 17, No. 2, pages 339-351, and in Lu et al. (2016) Int. J. Mol. Sci. 17, 561 (doi:10.3390/ijms17040561) the content of which is incorporated herein by reference.

Linkers can comprise a variety of chemical groups which include, but are not limited to, optionally substituted divalent radicals such as alkylene, arylene, heteroarylene; moieties such as: —(CR₂)ₙO(CR₂)ₙ—, repeating units of alkyloxy (e.g. polyethylenoxy, PEG, polymethyleneoxy) and alkylamino (e.g. polyethyleneamino); and diacid ester and amides including succinate, succinamide, diglycolate, malonate, and caproamide; wherein each R is independently H, C₁-C₁₈alkyl, C₆-C₂₀ aryl, C₃-C₁₄ heterocycle, a protecting group or a prodrug moiety, and n is an integer from 1 to 10.

In a specific embodiment, the linker may be peptidic, comprising one or more amino acid units. Examples of amino acid linkers include a dipeptide, a tripeptide, a tetrapeptide or a pentapeptide. Amino acid linker components may or may not be designed and optimized in their selectivity for enzymatic cleavage by a particular enzyme.

The second end of the first linker preferably comprises or consists of an iron complexing group which is capable of binding to the ferric iron of the ADC. The iron complexing group is preferably a chelating group. More preferably, the iron complexing group is a chelating group of a siderophore. In a preferred embodiment, the iron complexing group is a catecholate group, a carboxylate group, or a hydroxamate group, as shown in the following formulae (I) to (III), respectively, wherein R is the rest of the linker including the first end.

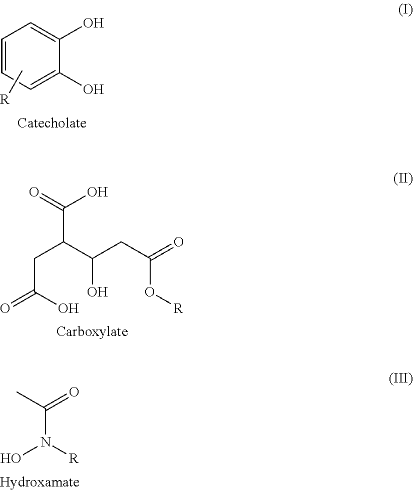

As used herein, the terms catecholate group, carboxylate group, and hydroxamate group include the respective acid forms and deprotonated forms thereof.

A preferred first linker of the ADC of the invention is a compound of formula (I), wherein R is —(CH₂)ₚ—CH(NHR²)—(C═O) . . . , p is an integer from 0 to 6, R² is a formyl group, an acetyl group, a peptidyl group, or a C₁-C₂₀ hydrocarbon group, and wherein the dotted line indicates the binding site to the antibody. Preferably, p is an integer from 2 to 5, more preferably p is 3 or 4, most preferably p is 3.

A particular first linker of the ADC of the invention is a compound of formula (I), wherein R is —(CH₂)ₚ—CH(NH₂)—(C═O) . . . , p is an integer from 0 to 6, and the dotted line indicates the binding site to the antibody. Preferably, p is an integer from 2 to 5, more preferably p is 3 or 4, most preferably p is 3.

Another preferred first linker of the ADC of the invention is a compound of formula (I), wherein R is —C═O—NH—(CH₂)ₚ—CH(NHR²)—(C═O) . . . , p is an integer from 0 to 6, R² is a formyl group, an acetyl group, a peptidyl group, or a C₁-C₂₀ hydrocarbon group, and the dotted line indicates the binding site to the antibody. Preferably, p is an integer from 2 to 5, more preferably p is 3 or 4, most preferably p is 3.

Another particular first linker of the ADC of the invention is a compound of formula (I), wherein R is —C═O—NH—(CH₂)ₚ—CH(NH₂)—(C═O) . . . , p is an integer from 0 to 6, and the dotted line indicates the binding site to the antibody. Preferably, p is an integer from 2 to 5, more preferably p is 3 or 4, most preferably p is 3.

A preferred first linker of the ADC of the invention is a compound of formula (II), wherein R is —(CH₂)ₚ—CH(NHR²)—(C═O) . . . , p is an integer from 0 to 6, R² is a formyl group, an acetyl group, a peptidyl group, or a C₁-C₂₀ hydrocarbon group, and wherein the dotted line indicates the binding site to the antibody. Preferably, p is an integer from 2 to 5, more preferably p is 3 or 4, most preferably p is 3.

A particular first linker of the ADC of the invention is a compound of formula (II), wherein R is —(CH₂)ₚ—CH(NH₂)—(C═O) . . . , p is an integer from 0 to 6, and the dotted line indicates the binding site to the antibody. Preferably, p is an integer from 2 to 5, more preferably p is 3 or 4, most preferably p is 3.

Another preferred first linker of the ADC of the invention is a compound of formula (II), wherein R is —C═O—NH—(CH₂)ₚ—CH(NHR²)—(C═O) . . . , p is an integer from 0 to 6, R² is a formyl group, an acetyl group, a peptidyl group, or a C₁-C₂₀ hydrocarbon group, and the dotted line indicates the binding site to the antibody. Preferably, p is an integer from 2 to 5, more preferably p is 3 or 4, most preferably p is 3.

Another particular first linker of the ADC of the invention is a compound of formula (II), wherein R is —C═O—NH—(CH₂)ₚ—CH(NH₂)—(C═O) . . . , p is an integer from 0 to 6, and the dotted line indicates the binding site to the antibody. Preferably, p is an integer from 2 to 5, more preferably p is 3 or 4, most preferably p is 3.

A preferred first linker of the ADC of the invention is a compound of formula (III), wherein R is —(CH₂)ₚ—CH(NHR²)—(C═O) . . . , p is an integer from 0 to 6, R² is a formyl group, an acetyl group, a peptidyl group, or a C₁-C₂₀ hydrocarbon group, and wherein the dotted line indicates the binding site to the antibody. Preferably, p is an integer from 2 to 5, more preferably p is 3 or 4, most preferably p is 3.

A particular first linker of the ADC of the invention is a compound of formula (III), wherein R is —(CH₂)ₚ—CH(NH₂)—(C═O) . . . , p is an integer from 0 to 6, and the dotted line indicates the binding site to the antibody. Preferably, p is an integer from 2 to 5, more preferably p is 3 or 4, most preferably p is 3.

Another preferred first linker of the ADC of the invention is a compound of formula (III), wherein R is —C═O—NH—(CH₂)ₚ—CH(NHR²)—(C═O) . . . , p is an integer from 0 to 6, R² is a formyl group, an acetyl group, a peptidyl group, or a C₁-C₂₀ hydrocarbon group, and the dotted line indicates the binding site to the antibody. Preferably, p is an integer from 2 to 5, more preferably p is 3 or 4, most preferably p is 3.

Another particular first linker of the ADC of the invention is a compound of formula (III), wherein R is —C═O—NH—(CH₂)ₚ—CH(NH₂)—(C═O) . . . , p is an integer from 0 to 6, and the dotted line indicates the binding site to the antibody. Preferably, p is an integer from 2 to 5, more preferably p is 3 or 4, most preferably p is 3.

In other preferred embodiments of the invention the iron complexing group is a pyridine derivative which is capable of complexing a ferric iron as defined hereinabove. Suitable pyridine derivatives are described in, e.g. “The Chemistry of Heterocyclic Compounds, Pyridine Metal Complexes”, ed. Desmond J. Brown, John Wiley & Sons, 2009 (ISBN-13: 9780470239728), the disclosure of which is incorporated herein in its entirety. The embodiments described above in connection with Formula (I), (II) or (III) are applicable to other chelating agent described in this reference mutatis mutandis.

Further suitable iron complexing groups are described in “Chelating Agents and Metal Chelates”, ed. F Dwyer, 2012 (ISBN-13: 9780323146418), the disclosure of which is incorporated herein in its entirety. The embodiments described above in connection with Formula (I), (II) or (III) are applicable to other chelating agent described in this reference mutatis mutandis.

The average number of first linkers per antibody (i.e. the molar “first linker-to-antibody ratio”) in the ADC of the invention is typically at least 1, preferably at least 2, more preferably at least 5, more preferably at least 8, more preferably at least 10, most preferably >10.

The molar “first linker-to-antibody ratio” in the ADC of the invention is typically 1 to 50, preferably 2 to 40, more preferably 5 to 35, more preferably 8 to 30, most preferably 10 to 20, e.g. 11 to 20, 12 to 20, 13 to 20, 14 to 20, or 15 to 20. In other embodiments the molar “first linker-to-antibody ratio” is 10 to 19, or 11 to 18, or 12 to 17, or 13 to 16, e.g.13, 14, 15 or 16.

The molar “first linker-to-antibody ratio” in the ADC of the invention is preferably >10.

The ADC of the present invention can comprise one or more ferric irons per antibody molecule. The number of ferric irons per antibody molecule (i.e. the molar “ferric iron-to-antibody ratio”) is preferably equal to the number of first linkers that were conjugated to the antibody, in case the first linker is capable of complexing only one ferric iron. Depending on the chemical design, the first linker may be capable of complexing more than one ferric iron, e.g. 2 or 3 ions (see Formula below).

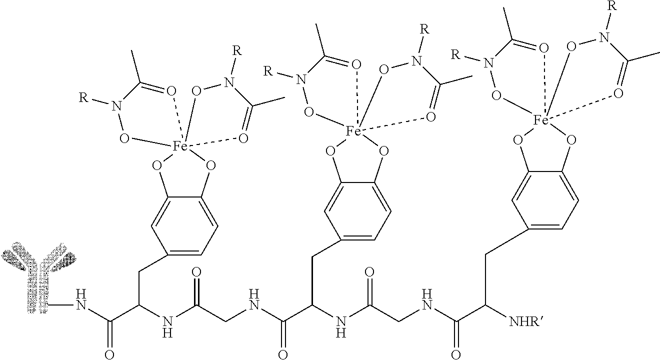

The average number of drug molecules per antibody (i.e. the molar “drug-to-antibody ratio”) in the ADC of the invention usually is twice the number of first linkers that are conjugated to the antibody. Hence, typically from 2 to 30 or from 4 to 20. In the example in the formula above, one first linker serves to complex 3 ferric ions, with 2*3=6 concomitant drug molecules. In the ADC of the invention the drug molecule is directly or indirectly bound to the ferric iron. The binding is preferably effected via iron complexing groups as defined above for the first linker.

If the drug molecule is directly bound to the ferric iron, the drug molecule itself is capable of binding to the ferric iron. According to this embodiment the drug molecule preferably comprises an iron complexing group. The iron complexing groups may or may not be the same as defined above for the first linker. Examples of drug molecules that are capable of binding to the ferric iron include the following HDAC inhibitors:

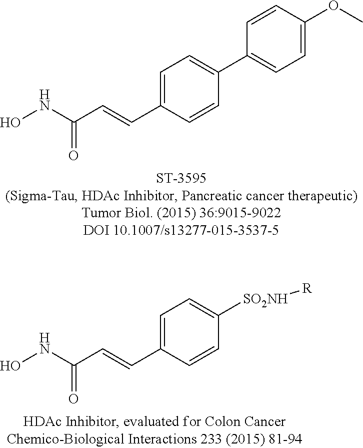

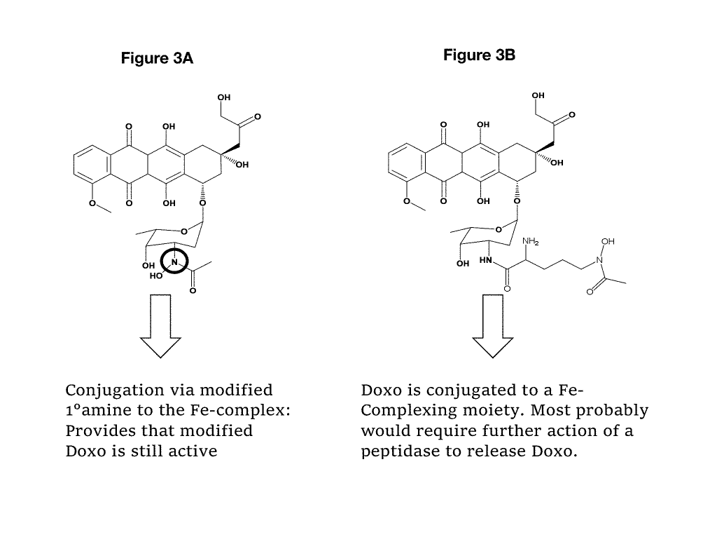

As can be seen, the drug molecules comprise a hydroxamate group which is capable of binding to a ferric iron. This is also illustrated in FIGS. 2A and 2B. FIG. 3A also shows an embodiment where the drug molecule (doxorubicin) is modified to allow binding to the ferric iron. As the released modified drug molecule is active, this is still considered “direct binding” of the drug molecule to the ferric iron.

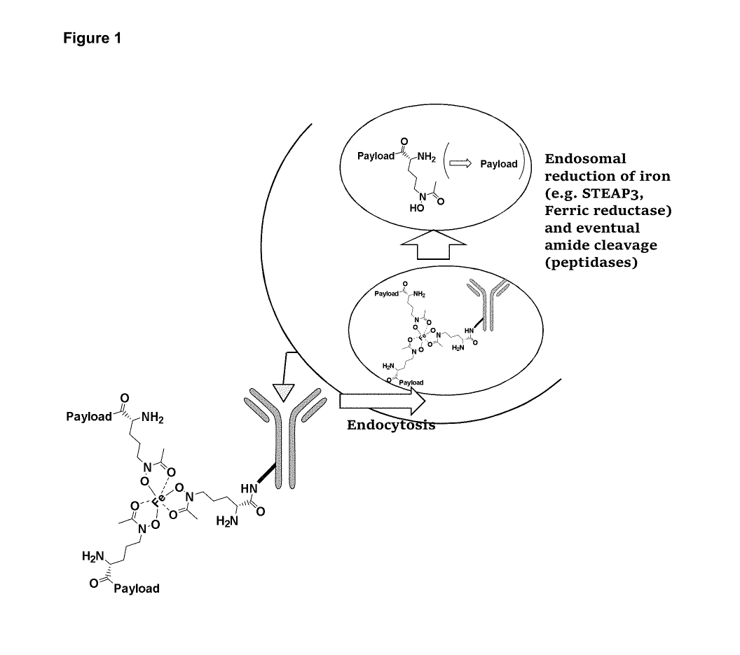

If the drug molecule is indirectly bound to the ferric iron the drug molecule is attached to a second linker which comprises a chemical group that is capable of binding to the ferric iron. The chemical group that is capable of binding to the ferric iron typically is a metal complexing group that is capable of binding to the ferric iron. The preferred iron complexing groups described above in connection with the first linker are also preferred iron complexing groups of the second linker. That is, one end of the second linker preferably comprises or consists of a hydroxamate group, a catecholate group or a carboxylate group. The other end of the second linker is preferably covalently attached to the drug molecule. This embodiment is illustrated in FIG. 1, wherein the drug molecule is designated “payload”. FIG. 3B also shows an embodiment where doxorubicin is attached to a linker which comprises an iron complexing group so as to allow binding to the ferric iron.

A preferred second linker of the ADC of the invention is a compound of formula (I), wherein R is —(CH₂)ₚ—CH(NHR²)—(C═O) . . . , p is an integer from 0 to 6, R² is a formyl group, an acetyl group, a peptidyl group, or a C₁-C₂₀ hydrocarbon group, and wherein the dotted line indicates the binding site to the drug molecule. Preferably, p is an integer from 2 to 5, more preferably p is 3 or 4, most preferably p is 3.

A particular second linker of the ADC of the invention is a compound of formula (I), wherein R is —(CH₂)ₚ—CH(NH₂)—(C═O) . . . , p is an integer from 0 to 6, and the dotted line indicates the binding site to the drug molecule. Preferably, p is an integer from 2 to 5, more preferably p is 3 or 4, most preferably p is 3.

Another preferred second linker of the ADC of the invention is a compound of formula (I), wherein R is —C═O—NH—(CH₂)ₚ—CH(NHR²)—(C═O) . . . , p is an integer from 0 to 6, R² is a formyl group, an acetyl group, a peptidyl group, or a C₁-C₂₀ hydrocarbon group, and the dotted line indicates the binding site to the drug molecule. Preferably, p is an integer from 2 to 5, more preferably p is 3 or 4, most preferably p is 3.

Another particular second linker of the ADC of the invention is a compound of formula (I), wherein R is —C═O—NH—(CH₂)ₚ—CH(NH₂)—(C═O) . . . , p is an integer from 0 to 6, and the dotted line indicates the binding site to the drug molecule. Preferably, p is an integer from 2 to 5, more preferably p is 3 or 4, most preferably p is 3.

A preferred second linker of the ADC of the invention is a compound of formula (II), wherein R is —(CH₂)ₚ—CH(NHR²)—(C═O) . . . , p is an integer from 0 to 6, R² is a formyl group, an acetyl group, a peptidyl group, or a C₁-C₂₀ hydrocarbon group, and wherein the dotted line indicates the binding site to the drug molecule. Preferably, p is an integer from 2 to 5, more preferably p is 3 or 4, most preferably p is 3.

A particular second linker of the ADC of the invention is a compound of formula (II), wherein R is —(CH₂)ₚ—CH(NH₂)—(C═O) . . . , p is an integer from 0 to 6, and the dotted line indicates the binding site to the drug molecule. Preferably, p is an integer from 2 to 5, more preferably p is 3 or 4, most preferably p is 3.

Another preferred second linker of the ADC of the invention is a compound of formula (II), wherein R is —C═O—NH—(CH₂)ₚ—CH(NHR²)—(C═O) . . . , p is an integer from 0 to 6, R² is a formyl group, an acetyl group, a peptidyl group, or a C₁-C₂₀ hydrocarbon group, and the dotted line indicates the binding site to the drug molecule. Preferably, p is an integer from 2 to 5, more preferably p is 3 or 4, most preferably p is 3.

Another particular second linker of the ADC of the invention is a compound of formula (II), wherein R is —C═O—NH—(CH₂)ₚ—CH(NH₂)—(C═O) . . . , p is an integer from 0 to 6, and the dotted line indicates the binding site to the drug molecule. Preferably, p is an integer from 2 to 5, more preferably p is 3 or 4, most preferably p is 3.

A preferred second linker of the ADC of the invention is a compound of formula (III), wherein R is —(CH₂)ₚ—CH(NHR²)—(C═O) . . . , p is an integer from 0 to 6, R² is a formyl group, an acetyl group, a peptidyl group, or a C₁-C₂₀ hydrocarbon group, and wherein the dotted line indicates the binding site to the drug molecule. Preferably, p is an integer from 2 to 5, more preferably p is 3 or 4, most preferably p is 3.

A particular second linker of the ADC of the invention is a compound of formula (III), wherein R is —(CH₂)ₚ—CH(NH₂)—(C═O) . . . , p is an integer from 0 to 6, and the dotted line indicates the binding site to the drug molecule. Preferably, p is an integer from 2 to 5, more preferably p is 3 or 4, most preferably p is 3.

Another preferred second linker of the ADC of the invention is a compound of formula (III), wherein R is —C═O—NH—(CH₂)ₚ—CH(NHR²)—(C═O) . . . , p is an integer from 0 to 6, R² is a formyl group, an acetyl group, a peptidyl group, or a C₁-C₂₀ hydrocarbon group, and the dotted line indicates the binding site to the drug molecule. Preferably, p is an integer from 2 to 5, more preferably p is 3 or 4, most preferably p is 3.

Another particular second linker of the ADC of the invention is a compound of formula (III), wherein R is —C═O—NH—(CH₂)ₚ—CH(NH₂)—(C═O) . . . , p is an integer from 0 to 6, and the dotted line indicates the binding site to the drug molecule. Preferably, p is an integer from 2 to 5, more preferably p is 3 or 4, most preferably p is 3.

In one embodiment, the second linker of the ADC of the invention is a non-cleavable linker. Examples of non-cleavable linkers are maleimide-alkane linkers (e.g. maleimido-caproyl linkers) and maleimide-cyclohexane linkers.

In another embodiment, the second linker of the ADC of the invention is a cleavable linker. Cleavable linkers include chemically labile linkers and enzyme cleavable linkers. The chemically labile linker can be an acid-cleavable linker or a reducible linker. The acid-cleavable linker may comprise a hydrazone group. The reducible linker may comprise a disulfide group. The enzyme cleavable linker typically comprises a chemical group which can be cleaved or degraded by one or more lysosomal enzymes. Suitable groups include a valine-citrulline dipeptide group, a phenylalanine-lysine dipeptide group, and a β-glucuronide group (which can be cleaved by β-glucuronidase).

In a specific embodiment of the invention, the first linker is a non-cleavable linker, and the second linker is a cleavable linker. In another specific embodiment of the invention, the first linker is a non-cleavable linker, and the second linker is a non-cleavable linker. In another specific embodiment of the invention, the first linker is a cleavable linker, and the second linker is a cleavable linker. In another specific embodiment of the invention, the first linker is a cleavable linker, and the second linker is a non-cleavable linker.

The drug molecule is released from the ADC upon internalization of the ADC by the target cell, preferably a cancer cell. Without wishing to be bound by theory, it is believed that the release is enhanced by a low pH, and by reduction of the ferric iron, e.g. by the enzyme ferric reductase. Due to the lower pH inside endosomes and increased presence of ferric reductase, an Fe³⁺-based ADC complex should decompose inside the endosome, enhancing release or diffusion of the smaller ligands to the intracellular space.

In one embodiment the drug molecule is released from the ADC of the invention when the ADC is in the endosomal compartment of a cell, preferably a mammalian cell, most preferably a human cell.

Preferably, at least 25%, more preferably at least 50%, more preferably at least 75%, most preferably at least 95% of the drug molecules are released from the ADC upon reduction of the ferric iron to ferrous iron, as determined in an assay as described in Example 3.

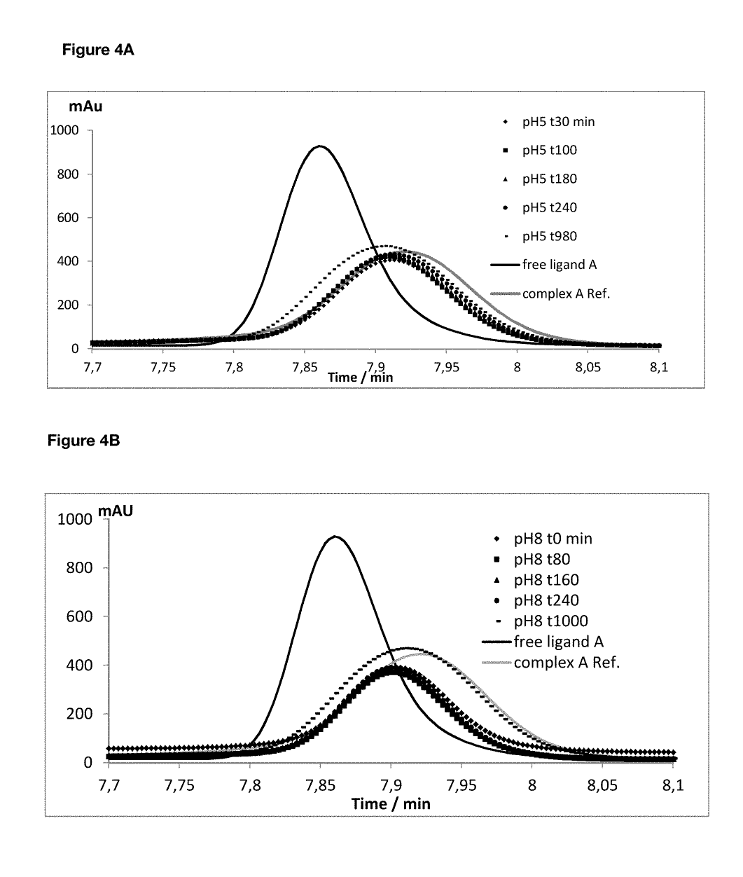

In another embodiment, the ADC of the invention is stable at a pH of 8, as determined in an assay as described in Example 1. In another embodiment, the ADC of the invention is stable at a pH of 7, as determined in an assay as described in Example 1. In another embodiment, the ADC of the invention is stable at a pH of 6, as determined in an assay as described in Example 1. In another embodiment, the ADC of the invention is stable at a pH of 5, as determined in an assay as described in Example 1. “Stable” in the sense of this assay means that incubation of the complex under the conditions of example 1 (pH 5 and pH 8) does not lead to release of free ligand which would be seen as a second peak in a graph according to FIGS. 4A and 4B.

Preferably, the drug molecule is not released from the ADC of the invention when the antibody is in blood, preferably in human blood. In other embodiments the drug molecule is not released from the ADC of the invention when the antibody is in blood plasma, preferably blood plasma from a vertebrate, more preferably human blood plasma.

In yet another embodiment, the ADC of the invention shows substantially no ligand exchange, as determined in an assay as described in Example 2.

Another aspect of the invention is an ADC having the following structure:

Ab[-L1-Me (-L2-D)ₙ]ₘ,
wherein
Ab is an antibody,
L1 is a first linker,
Me is ferric iron,
L2 is a second linker or absent,
D is a drug molecule,
m is from 1 to 10, and
n is from 1 to 3.

The preferred embodiments of antibody, first and second linker, ferric iron and drug molecule described above apply to this aspect of the invention mutatis mutandis. The parameter m is preferably from 2 to 6, or 3 to 5, e.g. 3, 4, or 5. The parameter n is preferably 2 or 3, most preferably 2. In a special embodiment m and/or n are/is an integer.

In a preferred embodiment n is 2 and m is 2.

In another preferred embodiment n is 2 and m is 3.

In another preferred embodiment n is 2 and m is 4.

In another preferred embodiment n is 2 and m is 5.

In another preferred embodiment n is 2 and m is 6.

In another preferred embodiment n is 2 and m is 7.

In another preferred embodiment n is 2 and m is 8.

In another preferred embodiment n is 2 and m is 9.

In another preferred embodiment n is 2 and m is 10.

In another preferred embodiment n is 3 and m is 2.

In another preferred embodiment n is 3 and m is 4.

In another preferred embodiment n is 3 and m is 5.

In another preferred embodiment n is 3 and m is 6.

In another preferred embodiment n is 3 and m is 7.

In another preferred embodiment n is 3 and m is 8.

In another preferred embodiment n is 3 and m is 9.

In another preferred embodiment n is 3 and m is 10.

Preparation of the ADC

Another aspect of the invention is a method for preparing the ADC of the present invention. The linkers can be synthesized by methods comprising steps that are known per se.

The ADC of the invention may be prepared as described in the following:

In the present invention, as described in example 5, an iron-complexing moiety is covalently attached to an antigen-recognizing structure (e.g. antibody) of interest. The most straightforward way is via succinimide activated conjugation as employed in example 5. However, a multitude of conjugation strategies are described in literature (e.g. maleinimide and click-chemistry methods). Following the conjugation of the first linker, the coupled product is purified and subsequently incubated respectively with a) two equivalents of a payload (this being an iron-complexing compound or any compound that is linked to an iron-complexing moiety) and b) one equivalent of an iron salt (in the case of example 5 this is FeCl₃). The resulting product is then purified again using a size exclusion column.

Treatment

In one embodiment, the invention provides a method of treating or preventing a disease comprising administering the ADCs of the invention to a patient, preferably a human patient. In certain embodiments, the disease to be treated or prevented is a cancer.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

For the prevention or treatment of disease, the appropriate dosage of an ADC will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 15 mg/kg (e.g. 0.1-20 mg/kg) of molecule is an initial candidate dosage for administration to the patient, whether, for example, by one or more separate administrations, or by continuous infusion. A typical daily dosage might range from about 1 μg/kg to 100 mg/kg or more, depending on the factors mentioned above. An exemplary dosage of ADC to be administered to a patient is in the range of about 0.1 to about 10 mg/kg of patient weight.

For repeated administrations over several days or longer, depending on the condition, the treatment is sustained until a desired suppression of disease symptoms occurs. An exemplary dosing regimen comprises administering an initial loading dose of about 4 mg/kg, followed by a weekly maintenance dose of about 2 mg/kg of the ADC. Other dosage regimens may be useful. The progress of this therapy is easily monitored by conventional techniques and assays.

Combination Therapy

An antibody-drug conjugate (ADC) may be combined in a pharmaceutical combination formulation, or dosing regimen as combination therapy, with a second compound having anti-cancer properties. The second compound of the pharmaceutical combination formulation or dosing regimen preferably has complementary activities to the ADC of the combination such that they do not adversely affect each other.

The second compound may be a chemotherapeutic agent, cytotoxic agent, cytokine, growth inhibitory agent, anti-hormonal agent, aromatase inhibitor, protein kinase inhibitor, lipid kinase inhibitor, anti-androgen, antisense oligonucleotide, ribozyme, gene therapy vaccine, anti-angiogenic agent and/or cardioprotectant. Such molecules are suitably present in combination in amounts that are effective for the purpose intended. A pharmaceutical composition containing an ADC may also have a therapeutically effective amount of a chemotherapeutic agent such as a tubulin-forming inhibitor, a topoisomerase inhibitor, or a DNA binder.

Other therapeutic regimens may be combined with the administration of an anticancer agent identified in accordance with this invention. The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein there is a time period while both (or all) active agents simultaneously exert their biological activities.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

Also falling within the scope of this invention are the in vivo metabolic products of the ADC compounds described herein, to the extent such products are novel and unobvious over the prior art. Such products may result for example from the oxidation, reduction, hydrolysis, amidation, esterification, enzymatic cleavage, and the like, of the administered compound. Accordingly, the invention includes novel and unobvious compounds produced by a process comprising contacting a compound of this invention with a mammal for a period of time sufficient to yield a metabolic product thereof.

Metabolites include the products of in vivo cleavage of the ADC where cleavage of any bond occurs that links the drug moiety to the antibody. Metabolic cleavage may thus result in the naked antibody, or an antibody fragment. The antibody metabolite may be linked to a part, or all, of the linker. Metabolic cleavage may also result in the production a drug moiety or part thereof. The drug moiety metabolite may be linked to a part, or all, of the linker.

Administration of Antibody-Drug Conjugate Pharmaceutical Formulations

Therapeutic ADCs may be administered by any route appropriate to the condition to be treated. The ADC will typically be administered parenterally, i.e. infusion, subcutaneous, intramuscular, intravenous, intradermal, intrathecal, bolus, intratumor injection or epidural (Shire et al (2004) J. Pharm. Sciences 93(6):1390-1402). Pharmaceutical formulations of therapeutic antibody-drug conjugates (ADC) are typically prepared for parenteral administration with a pharmaceutically acceptable parenteral vehicle and in a unit dosage injectable form. An antibody-drug conjugate (ADC) having the desired degree of purity is optionally mixed with pharmaceutically acceptable diluents, carriers, excipients or stabilizers, in the form of a lyophilized formulation or an aqueous solution (Remington's Pharmaceutical Sciences (1980) 16th edition, Osol, A. Ed.).

Acceptable parenteral vehicles, diluents, carriers, excipients, and stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). For example, lyophilized anti-ErbB2 antibody formulations are described in WO 97/0480.

Pharmaceutical formulations of a therapeutic antibody-drug conjugate (ADC) may contain certain amounts of unreacted drug moiety (D), antibody-linker intermediate (Ab-L), and/or drug-linker intermediate (D-L), as a consequence of incomplete purification and separation of excess reagents, impurities, and by-products, in the process of making the ADC; or time/temperature hydrolysis or degradation upon storage of the bulk ADC or formulated ADC composition. For example, a formulation of the ADC may contain a detectable amount of free drug D. Alternatively, or in addition to, it may contain a detectable amount of drug-linker intermediate D-L. Alternatively, or in addition to, it may contain a detectable amount of the antibody, Ab. An exemplary formulation of may contain up to 10% molar equivalent of free drug.

The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semi permeable matrices of solid hydrophobic polymers containing the ADC, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile, which is readily accomplished by filtration through sterile filtration membranes.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Aqueous suspensions contain the active materials (ADC) in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The pharmaceutical compositions of ADC may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, an aqueous solution intended for intravenous infusion may contain from about 3 to 500 μg of the active ingredient per milliliter of solution in order that infusion of a suitable volume at a rate of about 30 mL/hr can occur. Subcutaneous (bolus) administration may be effected with about 1.5 ml or less of total volume and a concentration of about 100 mg ADC per ml. For ADC that require frequent and chronic administration, the subcutaneous route may be employed, such as by pre-filled syringe or autoinjector device technology.

As a general proposition, the initial pharmaceutically effective amount of ADC administered per dose will be in the range of about 0.01-100 mg/kg, namely about 0.1 to 20 mg/kg of patient body weight per day, with the typical initial range of compound used being 0.3 to 15 mg/kg/day. For example, human patients may be initially dosed at about 1.5 mg ADC per kg patient body weight. The dose may be escalated to the maximally tolerated dose (MTD). The dosing schedule may be about every 3 weeks, but according to diagnosed condition or response, the schedule may be more or less frequent. The dose may be further adjusted during the course of treatment to be at or below MTD which can be safely administered for multiple cycles, such as about 4 or more.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Although oral administration of protein therapeutics are generally disfavored due to poor bioavailability due to limited absorption, hydrolysis or denaturation in the gut, formulations of ADC suitable for oral administration may be prepared as discrete units such as capsules, cachets or tablets each containing a predetermined amount of the ADC.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Exemplary unit dosage formulations contain a daily dose or unit daily sub-dose, or an appropriate fraction thereof, of the active ingredient.

The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

A further aspect of the present invention is the use of the ADC described herein for preventing or reducing the release of a drug molecule from an ADC in blood or plasma, preferably in human blood or plasma. Another aspect of the invention is the use of ferric iron for preventing or reducing the release of a drug molecule from an antibody-drug conjugate in blood or plasma, preferably in human blood or plasma, wherein the ferric iron is complexed by the antibody and the drug molecule to form the antibody-drug conjugate. Another aspect of the invention is a method of preventing or reducing the release of a drug molecule from an antibody-drug conjugate in blood, comprising complexing ferric iron with the antibody and the drug molecule to form the antibody-drug conjugate. The preferred embodiments of the uses and methods of the invention correspond to those of the ADC of the invention as described hereinabove.

### EXAMPLES

### Example 1

To assess the stability of the complexes that we intended to use for the above described linkers, a number of experiments were conducted. To this end, two model-ligands (GVFe35 and ST088) were synthesized (See Scheme 1).

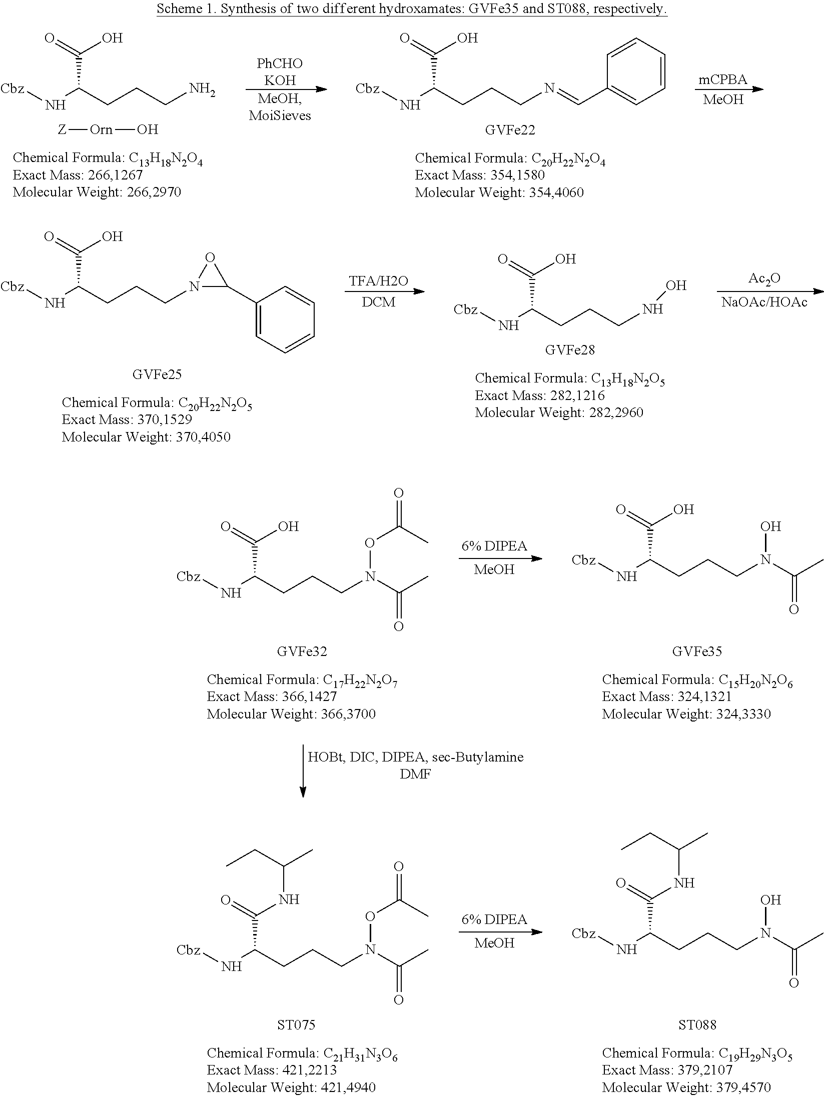

Using these two ligands, the following two model complexes were prepared by addition of FeCl₃ solution.

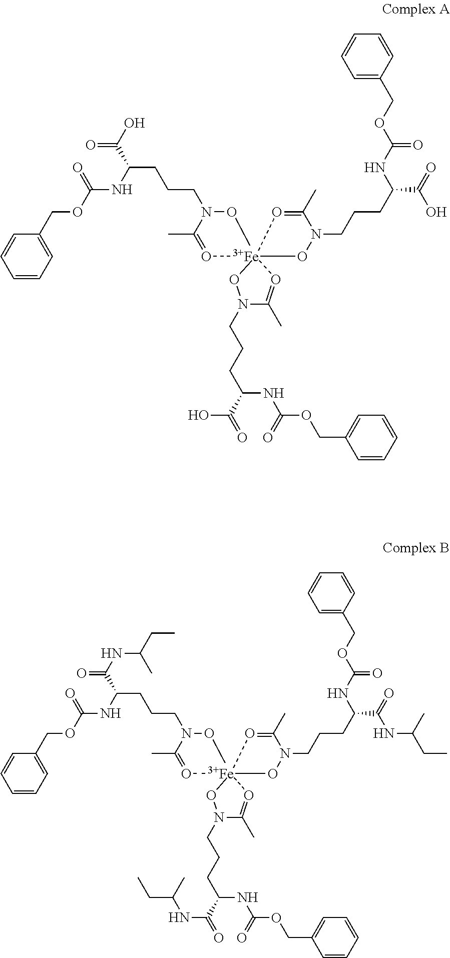

The formulae show the structures of iron complexes formed using GVFe35 (free carbonic acid, Complex A) and ST088 (sec-butylamine coupled, Complex B).

Assessing the complex stability at different pHs over time, Complex A was then incubated at pH 5.0 or pH 8.0 at 37° C. Eventual degradation of the complex was monitored by analytical HPLC at regular time-intervals and after 16 h. The HPLC-chromatograms are depicted in FIGS. 4A and 4B. It follows, that after 16 h only modest degradation could be observed in both cases, indicating that under these conditions the complexes are stable.

### Example 2

To examine if ligand exchange occurs (i.e. ligand dissociation and renewed complexation), an additional experiment was conducted, whereby both complexes A and B were mixed and incubated at 37° C. at pH 7.0 for 16 h. In case of dissociation and subsequent complexation one would expect intermediate complexes, whereby one or two ligands of complex B would complex iron together with one ligand stemming from complex A or vice versa.

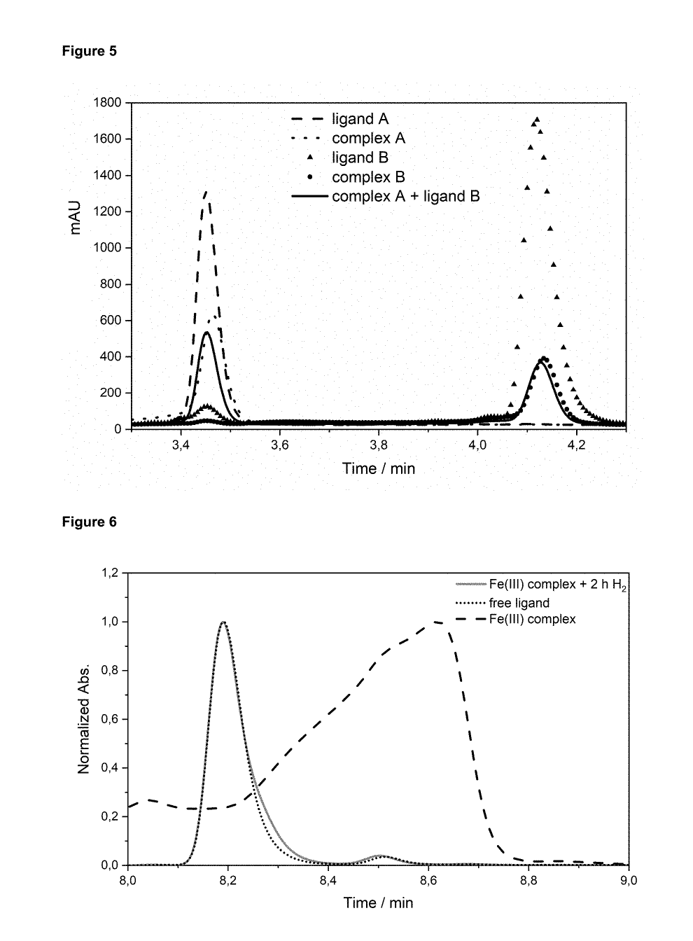

Both complexes and the mixture were examined by analytical HPLC (See FIG. 5). Due to the more apolar sec-butyl amide function in ST088, that was used for complex B, this complex has a longer retention time than complex A. Therefore, in case of ligand exchange, peaks with intermediate retention time would start occurring between the two complex peaks. Since no intermediate peaks were observed, this suggests that no ligand exchange occurs for the examined complexes, indicating high stability.

### Example 3

To further corroborate this result and to examine the influence of the irons oxidation state, further experiments were conducted. Free ligand was mixed with FeCl₃, giving complex A that was subsequently reduced by hydrogenation:

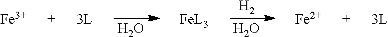

Ligand, complex A, and reduced material were analyzed by analytical LC (See FIG. 6). It is shown that complex A has a longer retention time than the free ligand. However, after reduction, only a peak with the same retention time as the native ligand shows. From this, it must be concluded that the reduced complex is destabilized, leading to decomplexation which in turn results in un-complexed ferrous iron and the free ligands. A complementary experiment showed respective results for the oxidation of Fe(II) in the presence of free ligand and air.

### Example 4

For further studies of antibodies to which iron complexes are conjugated, a fluorescently Rhodamine 101) labeled derivative of a lysine based hydroxamate was synthesized (see Scheme 2).

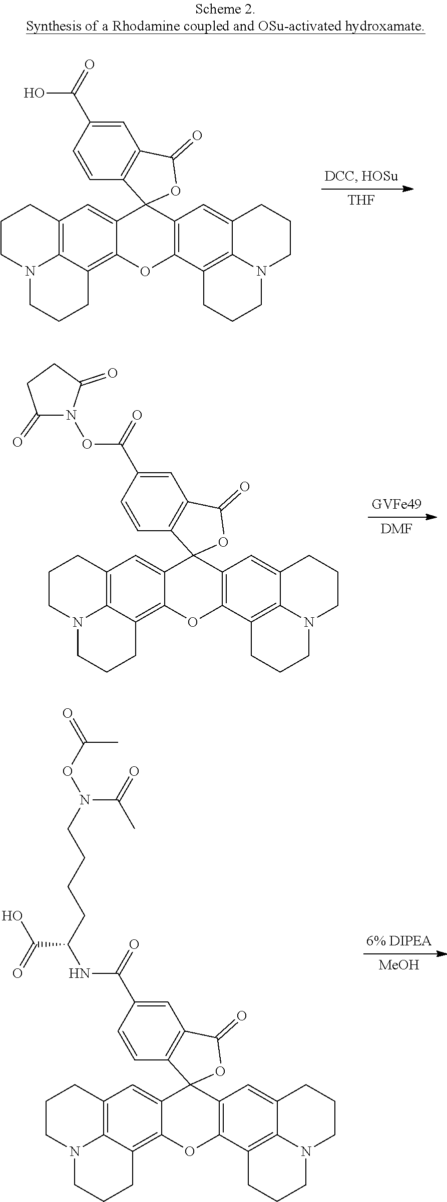

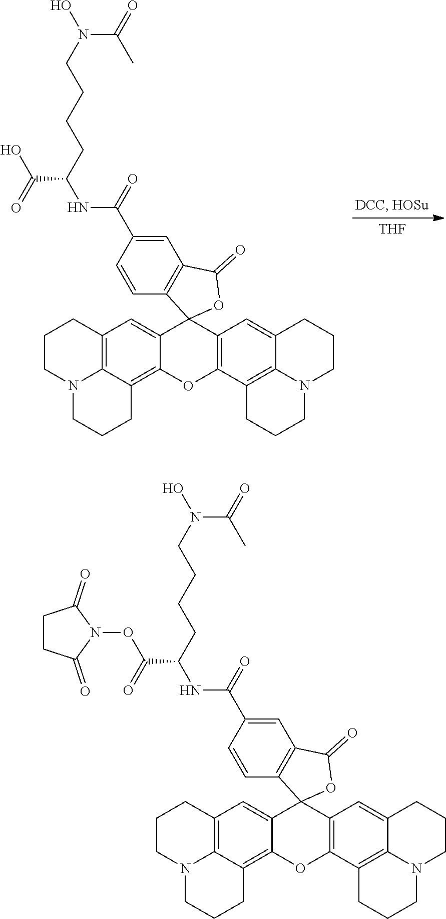 [SMILES: CC(=O)N(O)CCCC[C@H](NC(=O)c1ccc2c(c1)C(=O)OC21c2cc3c4c(c2Oc2c1cc1c5c2CCCN5CCC1)CCCN4CCC3)C(=O)O ; "THF" ; CC(=O)N(O)CCCC[C@H](NC(=O)c1ccc2c(c1)C(=O)OC21c2cc3c4c(c2Oc2c1cc1c5c2CCCN5CCC1)CCCN4CCC3)C(=O)ON1C(=O)CCC1=O]

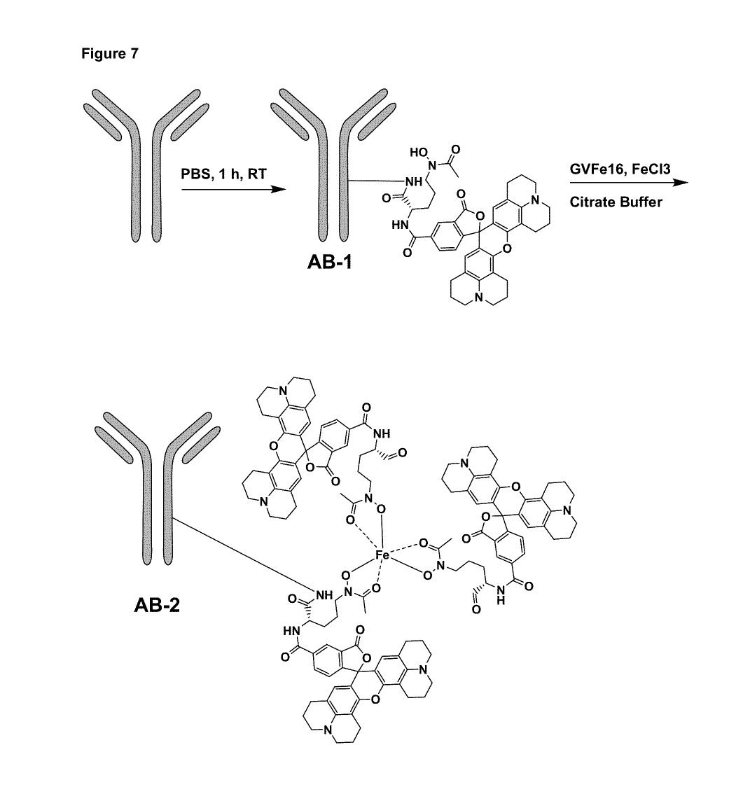

This ligand was next conjugated to Trastuzumab for further complexation together with the non-OSu activated ligand, which is also coupled to Rhodamine 101 (see FIG. 7).

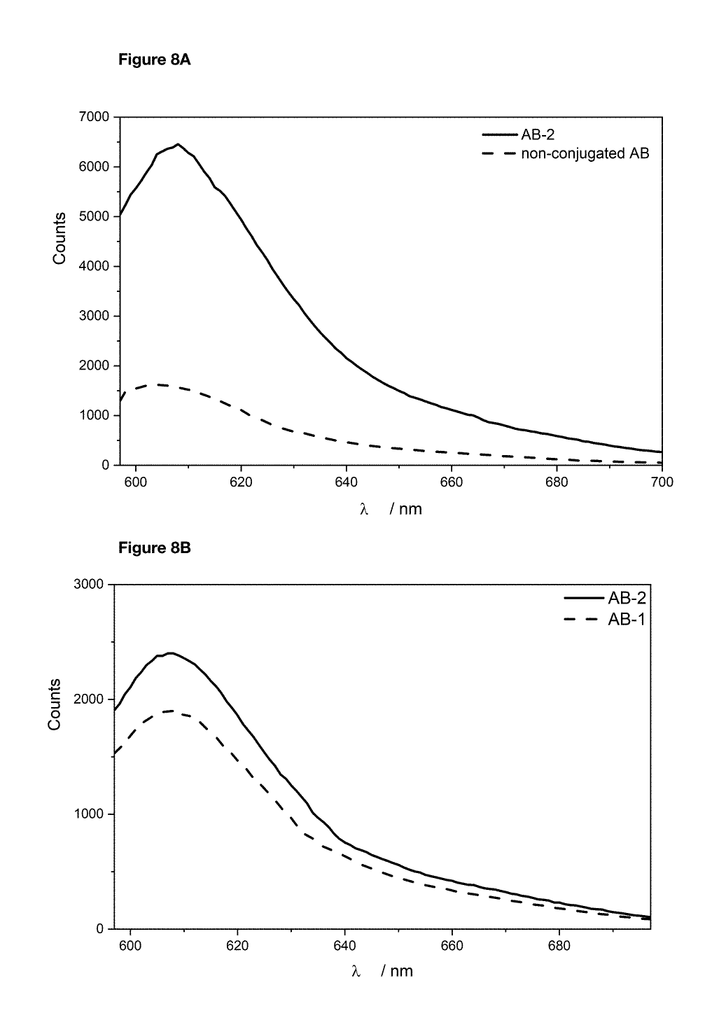

The AB-conjugated complex was purified by centrifugation in Vivaspin tubes (cut-off <30 kDa), by washing the conjugated complex twice with citrate buffer (5 mL). This was followed by diluting the product to 0.5 mL and fluorescence measurement. From FIGS. 8A and 8B, it follows, that AB-2 yields more fluorescence than the non-conjugated AB, and AB-1 less than AB-2. A significant difference in fluorescence for both compounds is observed, showing that complexation with iron and the two auxiliary ligands was successful (see FIG. 8B).

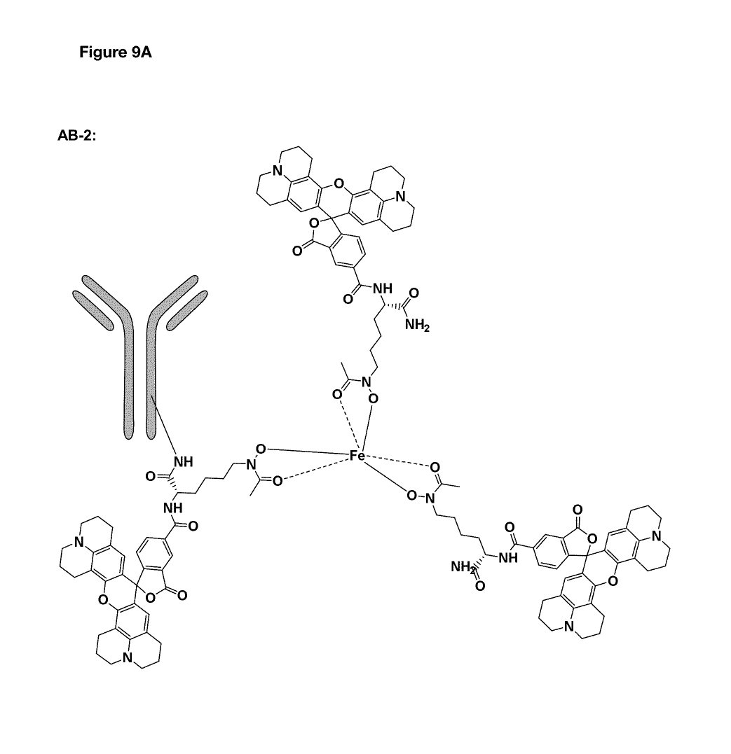

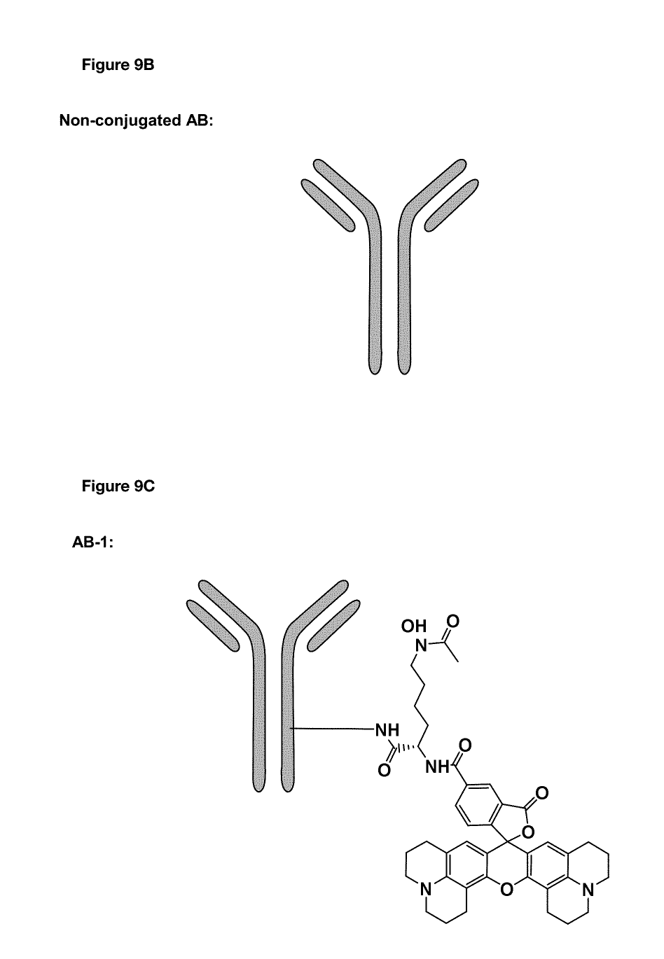

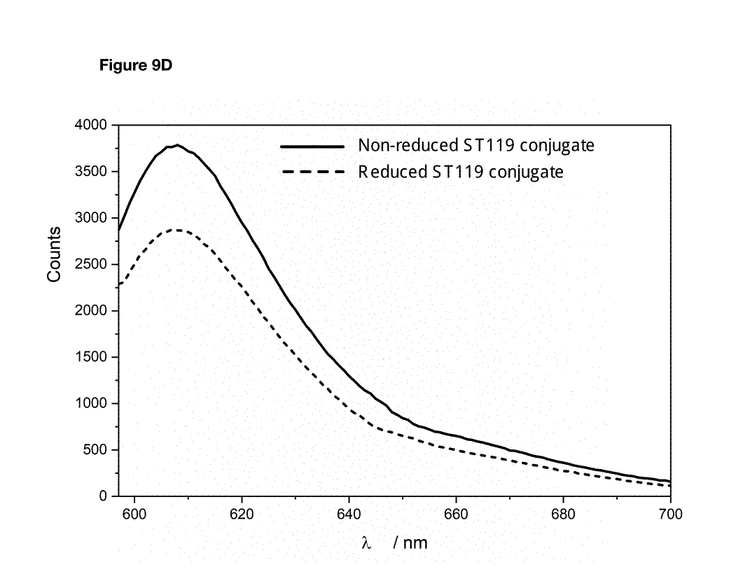

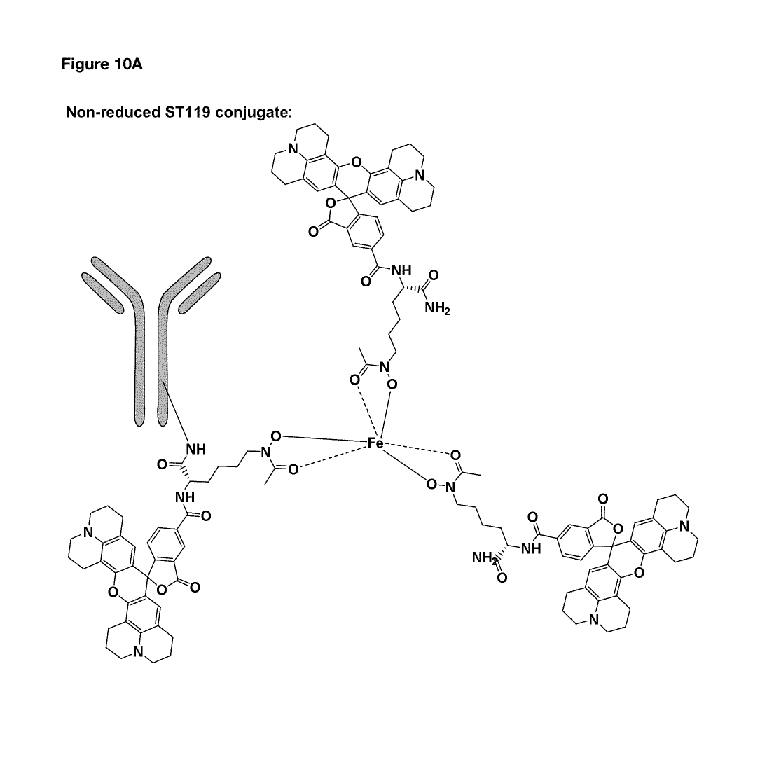

To examine if the complex can disintegrate by destabilization due to reduction of the ferric form of the complex into the ferrous complex, AB-2 (FIG. 9A) was reduced to obtain AB-1 (FIG. 9C). To this end, AB-2 was reduced under hydrogen atmosphere for 1 h at RT and purified by Vivaspin centrifugation and washed twice with 5 mL citrate buffer. This was followed by fluorescence measurement (see FIG. 9D), from which it can be observed that the control, that underwent the same procedure (except hydrogenation), shows again ˜20% more fluorescence than the reduced product. This shows that AB-2 was reduced to give AB-1.

### Example 5

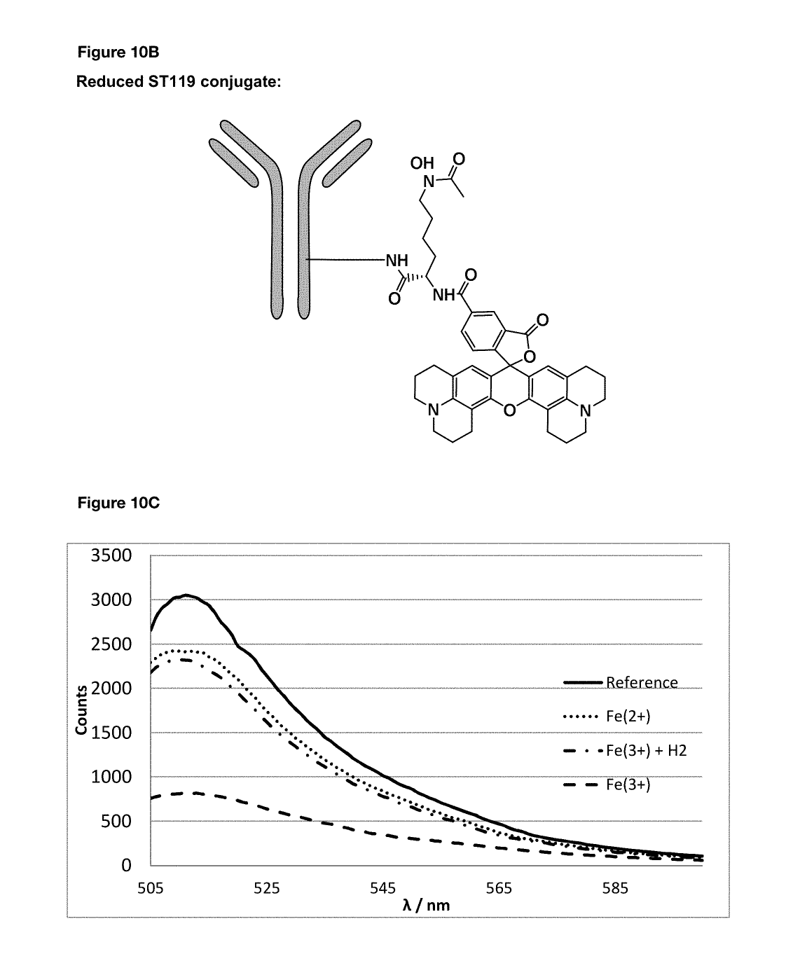

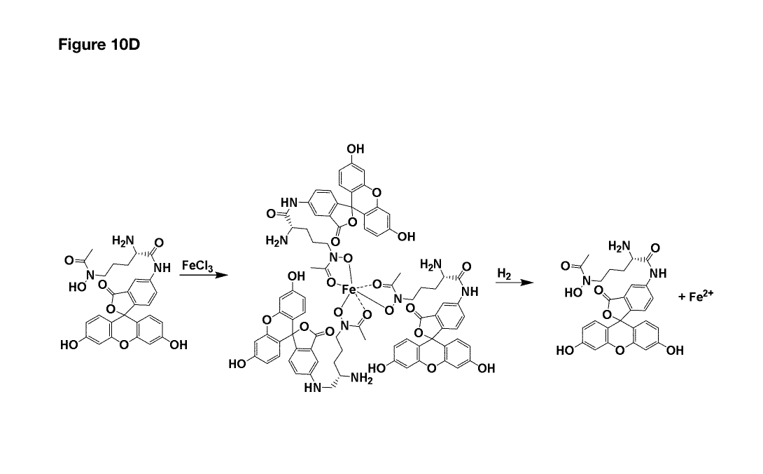

To assess whether an antibody conjugate, containing a payload that is bound via ferric iron to the antybody, would a) actually be endocytosed into cells expressing the appropriate antigen, and b) retains sufficient stability in proximity of cell membranes in physiological medium, a trastuzumab conjugate as depicted in Scheme 3 was prepared. As a first linker, a lysine-based hydroxamate that was also conjugated to BODIPY was used. The hydroxamate moiety of this structure is able to complex ferric iron with two auxiliary ligands. As auxiliary ligands, again lysine-based hydroxamates were used that in this case were conjugated to Rhodamine 101. Hence, a trastuzumab conjugate was created that contained a covalently linked BODIPY tag, capable of complexing ferric iron, as well as two Rhodamine containing ligands that are non-covalently conjugated to the antibody via the ferric iron complex. See FIGS. 10A and 10B.

SK-BR3-Cells were grown overnight on coverslips with Polyornithincoating. Subsequently these cells were incubated with GVFe66b up to 30 minutes or 6 h and washed twice with PBS buffer and fixated (PFA).

Fluorescence microscopy after 30 minutes and six hours shows both the BODIPY and rhodamine signal on the cell surface after one hour and in endosomal compartments after six hours. From this observation it must be concluded that the conjugate retains certain stability up to internalization. See FIG. 10C.

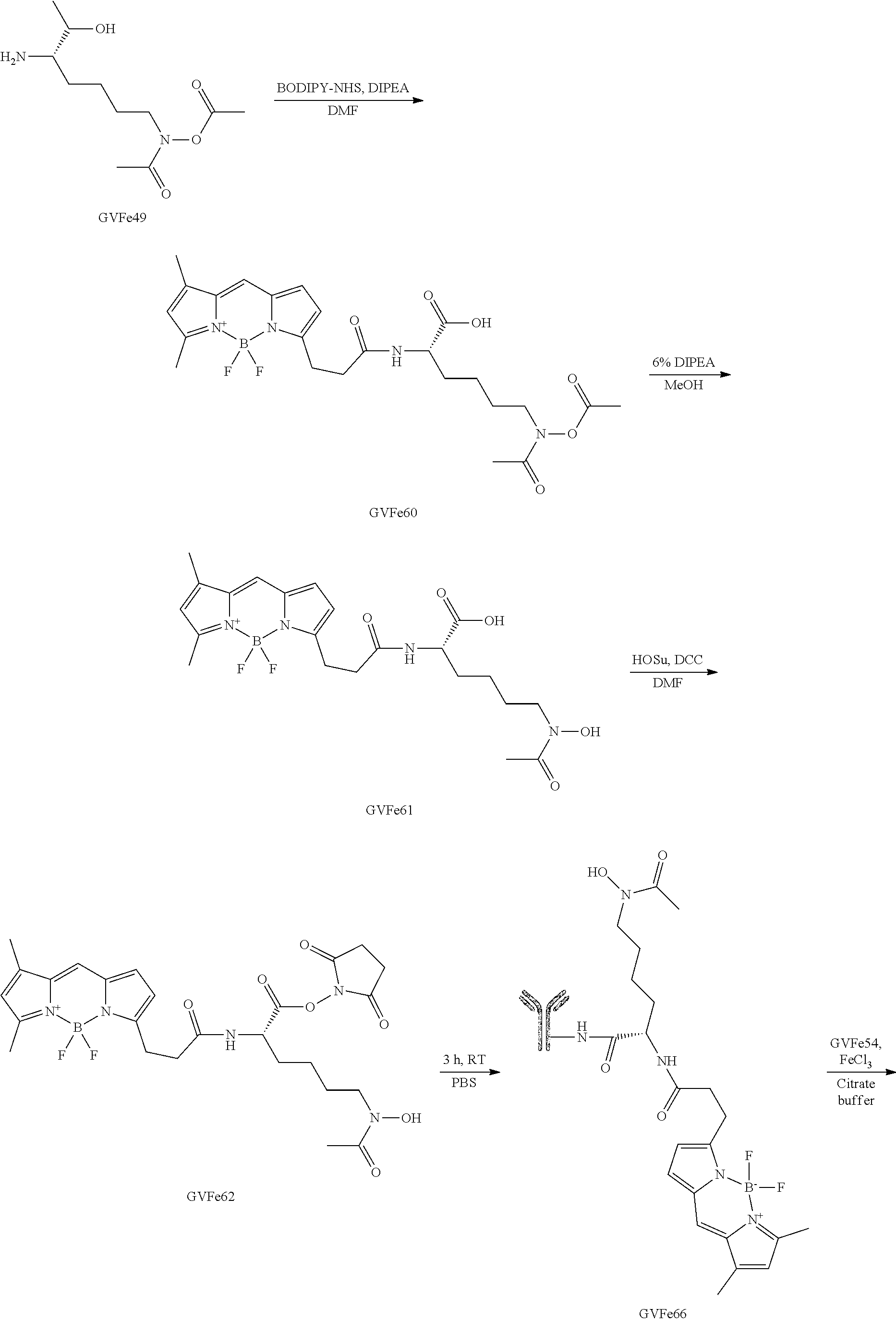

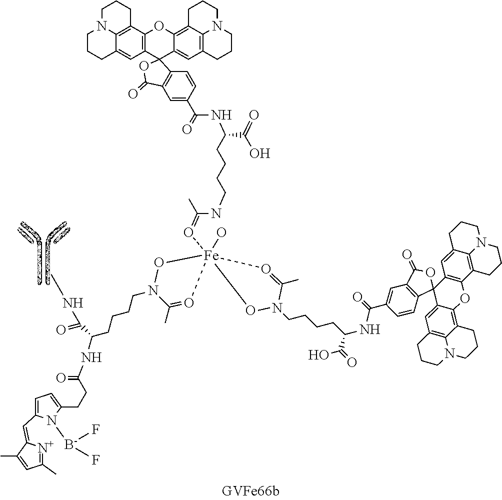

Scheme 3. Preparation of a Trastuzumab conjugated ADC, using a ferric iron linker concept. Compound GVFe49 was prepared by standard literature based synthesis. Subsequently, GVFe49 was conjugated to commercially available BODIPY-NHS, yielding compound GVFe60, which was next deprotected, yielding GVFe61, and OSu activated, yielding GVFe62. This compound was next used to conjugate to the antibody. Subsequently, this conjugate was incubated with FeCl₃ and compound GVFe54 (which was in turn synthesized via the same method as described for compound GVFe49).

1. An antibody-drug conjugate comprising an antibody, at least one ferric iron bound to the antibody, and at least one drug molecule bound to the ferric iron.

2. The antibody-drug conjugate of claim 1, wherein the drug molecule is not released from the antibody-drug conjugate when the antibody-drug conjugate is in blood.

3. The antibody-drug conjugate of claim 1, wherein the drug molecule is released from the antibody-drug conjugate when the antibody-drug conjugate is in the endosomal compartment of a cell.

4. The antibody-drug conjugate of claim 1, wherein at least two drug molecules are bound to one ferric iron.

5. The antibody-drug conjugate of claim 1, wherein the average number of drug molecules per antibody in said conjugate is at least 10.

6. The antibody-drug conjugate of claim 1, wherein the drug molecule is an anti-cancer agent.

7. The antibody-drug conjugate of claim 1, wherein the antibody is capable of binding to an antigen expressed on a tumor cell.

8. The antibody-drug conjugate of claim 1, wherein the drug molecule comprises an iron complexing moiety that binds to the ferric iron.

9. The antibody-drug conjugate of claim 1, wherein the drug molecule is coupled to an iron complexing moiety which binds to the ferric iron.

10. The antibody-drug conjugate of claim 8, or wherein said iron complexing moiety is selected from the group consisting of a hydroxamate moiety, a catecholate moiety and a carboxylate moiety.

11. The antibody-drug conjugate of claim 1, having the structure:
Ab[-L1-Me (-L2-D)ₙ]ₘ,
wherein
Ab is the antibody,
L1 is a first linker,
Me is the ferric iron,
L2 is a second linker or absent,
D is the drug molecule,
m ranges from 1 to 10, and
n ranges from 1 to 3.

12. The antibody-drug conjugate of claim 1, wherein the drug molecule is released from the ferric iron if the ferric iron is reduced.

13. The antibody-drug conjugate of claim 1, wherein the drug molecule is released from the ferric iron at a pH of less than 5.

14. The antibody-drug conjugate of claim 1, which is stable at pH 8.

15. The antibody-drug conjugate of claim 1, which is stable at pH 5.

16. A pharmaceutical composition comprising the antibody-drug conjugate of claim 1.

17. A method of treating disease in a patient in need thereof, said method comprising the step of administering to said patient an effective amount of the antibody-drug conjugate of claim 1, or a pharmaceutical composition comprising said antibody-drug conjugate.

18. The method of claim 17, wherein said disease is cancer.

19. A method of preventing or reducing the release of a drug molecule from an antibody-drug conjugate in blood, said method comprising the step of complexing a ferric iron with said antibody and said drug molecule to form said antibody-drug conjugate.

20. The method of claim 19, wherein the drug molecule is released from the antibody-drug conjugate in the endosomal compartment of a cell in said patient.

21. The method of claim 19, wherein the antibody-drug conjugate is comprised of an antibody, at least one ferric iron bound to the antibody, and at least one drug molecule hound to the ferric iron.

22. (canceled)

23. (canceled)

24. (canceled)
